(19) United States
(12) Patent Application Publication
(10) Pub. No.: US 2015/0337315 A1
(43) Pub. Date: Nov. 26, 2015

(54) COMPOSITIONS AND METHODS TARGETING SIGNALING BY THE G PROTEIN BETA-GAMMA SUBUNIT FOR THE TREATMENT OF ASTHMA

(71) Applicant: Michael M. Grunstein, Studio City, CA (US)

(72) Inventor: Michael M. Grunstein, Studio City, CA (US)

(21) Appl. No.: 14/717,994

(22) Filed: May 20, 2015

Related U.S. Application Data
(60) Provisional application No. 62/000,634, filed on May 20, 2014.

(51) Int. Cl. C12N 15/113; A61K 31/713; A61K 45/06; A61K 9/00

(52) U.S. Cl. CPC C12N 15/1137; A61K 9/0073; A61K 31/713; A61K 45/06; C12N 2320/31; C12N 2310/14; C12N 2320/32

(57) ABSTRACT

Compositions and methods for the treatment of asthma are disclosed.

This application claims priority to U.S. Provisional Application No. 62/000,634 filed May 20, 2014, the entire contents being incorporated herein by reference as though set forth in full.

### FIELD OF THE INVENTION

This invention relates to the fields of signal transduction, respiratory physiology and the treatment or alleviation of the asthmatic condition. More specifically, the invention provides molecules which modulate G protein signaling which, upon delivery to the airway, alleviate the symptoms of asthma.

### BACKGROUND OF THE INVENTION

Several publications and patent documents are cited throughout the specification in order to describe the state of the art to which this invention pertains. Each of these citations is incorporated herein by reference as though set forth in full.

Heterotrimeric G proteins play crucial roles in regulating the asthmatic state, including the induction of airway hyper-responsiveness (AHR) and inflammation [1]. Upon activation by G protein-coupled receptors (GPCRs) responding to a host of bronchoactive and proinflammatory stimuli, the G protein α subunit undergoes an exchange of GDP for GTP and becomes dissociated from the βγ subunits [2], thereby allowing both free Gα and Gβγ to activate their respective effectors, notably including those that stimulate the MAPK signaling pathways. The latter regulate various aspects of the airway asthmatic response including immune and inflammatory cell functions [3], as well as airway smooth muscle (ASM) function, due to activation of transcription factors and other downstream molecules that mediate the release of proinflammatory cytokines, chemokines, and other molecules that can alter ASM contractility and proliferation [4-7]. In this regard, GPCR-dependent (also receptor-independent) stimulation of the Ras/c-Raf1/MEK signaling cascade leading to downstream activation of the MAPK, ERK1/2, characteristically uses signals generated by the βγ subunits of the pertussis toxin (PTX)-sensitive family of G proteins that inhibits adenylate cyclase activity (i.e., Gi proteins) via activation of the tyrosine kinase, c-Src [8-12]. This PTX-sensitive Gi protein-regulated mechanism was found to play a particularly important role in mediating the heightened constrictor and impaired relaxation responses exhibited in isolated ASM tissues exposed to various proasthmatic conditions including passive sensitization with serum from atopic asthmatic patients [13], proinflammatory cytokine exposure [14], inoculation with rhinovirus [15], and prolonged heterologous and homologous β2-adrenergic receptor (β2AR) desensitization [16,17]. In this connection, the altered responsiveness exhibited in β2AR-desensitized ASM was attributed to upregulated phosphodiesterase 4 (PDE4) activity induced by activation of the Gβγ subunit of Gi protein and its consequent activation of c-Src-induced signaling via the Ras/c-Raf1/MEK pathway leading to ERK1/2 activation, the latter eliciting transcriptional upregulation of the PDE4D5 subtype [16,17].

Recently, the above Gi-βγ-regulated mechanism implicated in mediating PDE4-dependent proasthmatic changes in contractility in β2AR-desensitized ASM was also found to mediate the in vivo airway hyperresponsiveness and inflammation elicited by inhaled antigen challenge in a rabbit model of allergic asthma [18]. In light of this evidence, together with that in recent studies demonstrating a pivotal role for PDE4 activity in regulating airway function in asthmatic individuals [19-21] and in animal models of allergic asthma [22-26], and that PDE4 activity is intrinsically increased in cultured human ASM (HASM) cells isolated from asthmatic individuals [27], the present study sought to determine whether asthmatic HASM cells exhibit constitutively increased PDE activity that is mechanistically attributed to intrinsically upregulated Gβγ signaling coupled to c-Src-induced activation of the Ras/MEK/ERK1/2 pathway. The results demonstrated that: 1) relative to normal (non-asthmatic) HASM cells, primary cultures of asthmatic HASM cells exhibit markedly increased constitutive PDE4 activity associated with free (activated) Gβγ-coupled c-Src and ERK1/2 activation; 2) this Gβγ-regulated increase in PDE activity is attributed to intrinsically enhanced co-localization of phosphorylated ERK1/2 with the PDE isoform, PDE4D, and 3) inhibition of Gβγ signaling acutely suppresses (within minutes) the increased PDE activity in asthmatic HASM cells to near normal levels, in association with suppression of c-Src and ERK1/2 activation, and co-localization of the latter with PDE4D. Finally, together with increased PDE activity attributed to free Gβγ-regulated ERK1/2 activation, the results demonstrated that asthmatic HASM cells also exhibit markedly increased direct binding of the small Rap1 GTPase-activating protein (Rap1GAP) to the α-subunit of G protein, a phenomenon that serves to cooperatively facilitate Ras-induced ERK1/2 activation, thereby enabling enhanced Gβγ-regulated PDE activity. Taken together, these new findings are the first to identify that asthmatic HASM cells exhibit constitutively increased PDE activity that is mechanistically attributed to intrinsically increased Gβγ signaling, facilitated by Rap1GAP recruitment to the Gα-subunit, leading to heightened c-Src-dependent/Ras-mediated activation of ERK1/2 and its consequent direct binding to and activation of PDE4. Given the crucial role attributed to upregulated PDE activity in the pathobiology of asthma, these new findings highlight a heretofore-unidentified decisive role for Gβγ signaling in regulating the heightened PDE4 activity that characterizes the asthmatic airway, and suggest that interventions targeted at suppressing Gβγ signaling associated with Gi protein activation may lead to novel approaches to treat asthma.

### SUMMARY OF THE INVENTION

The results presented herein demonstrate that heightened Gi-βγ signaling mediates the up-regulated PDE4 activity intrinsically detected in human airway smooth muscle (HASM) cells isolated from asthmatic patients and that this heightened Gi-βγ signaling is facilitated by intrinsically increased binding of Rap1GAP to the α-subunit of Gi protein in the asthmatic HASM cells. The results further demonstrate that, as with inhibitors of Gi-βγ signaling, inhibition of Rap1GAP suppresses the upregulated PDE4 activity in asthmatic HASM cells. Thus, in accordance with the present invention, compositions and methods are provided for delivering inhibitors of Rap1GAP activity into the airway of patients with asthma. The inhibitors can be siRNA, shRNA, antisense oligonucleotides or a small molecule that interferes with Rap1GAP activity. In preferred embodiments, the inhibitor acts directly on Rap1GAP expression or function and does not act via modulation of an upstream signaling agent. The compositions of the invention can be directly delivered to the lung, preferably in aerosolized form. They may optionally be combined with one or more conventional agents employed in the treatment of asthma, including without limitation, corticosteroids, sodium cromolyn, methylxanthines, phosphodiesterase inhibitors, beta 2 adrenergic agents, and leukotriene modifiers. They may also be combined with newly identified inhibitors of Gi-βγ signaling.

### BRIEF DESCRIPTION OF THE DRAWINGS

FIGS. 1A and 1B. Inhibition of Gβγ signaling suppresses intrinsically increased PDE activity in asthmatic HASM cells: Comparison of suppressive effects of anti-Gβγ blocking peptide vs. gallein. Relative to normal cells, asthmatic HASM cells exhibit significantly increased constitutive (baseline) PDE activity. Inhibition of Gβγ signaling with a maximal effective concentration (1 μM) of either anti-Gβγ blocking peptide (FIG. 1A) or gallein (FIG. 1B) acutely suppresses PDE activity in asthmatic HASM cells to normal levels at 0.25 hr. Contrasting sustained inhibition of PDE activity in cells treated with anti-Gβγ blocking peptide, suppression of PDE activity in asthmatic HASM cells treated with gallein is transient, with progressive recovery in PDE activity after 1 hr that approaches near basal elevated levels by 24 hr. Data are mean±SE values of 5-8 determinations made at each time point Comparisons are made using two-tailed Student t-test. *p<0.05; **p<0.01.

FIG. 2. Dose-dependent effects of anti-Gβγ blocking peptide on PDE activity in normal vs. asthmatic HASM cells. Relative to normal cells, basal PDE activity is significantly increased in asthmatic HASM cells. Unlike in normal cells wherein administration of anti-Gβγ blocking peptide has no effect, asthmatic HASM cells treated with increasing doses of anti-Gβγ blocking peptide (each dose treatment for 6 hr) exhibit significant dose-dependent suppression of PDE activity, with maximal suppressive effect attained using 1 μM. Data are mean±SE values. Comparisons of asthmatic HASM cells relative to basal normal levels are made using two-tailed Student t-test. *p<0.05; **p<0.01.

FIGS. 3A-3F. Gβγ-regulated c-Src and ERK1/2 activation is constitutively increased in asthmatic HASM cells and evoked in normal HASM cells stimulated with IL-13. (FIG. 3A) Western blot analysis demonstrating that, relative to normal cells, untreated asthmatic HASM cells exhibit constitutively increased free Gβ levels and correspondingly reduced co-localization of Gβ with immunoprecipitated Gα, denoting an intrinsic state of G protein activation. This pattern of Gβ distribution is acutely reversed in asthmatic HASM cells treated either with gallein or the anti-Gβγ blocking peptide (1 μM; ×30 min), By comparison, neither Gβγ inhibitor alters Gβ distribution in normal cells. (FIG. 3B) Immunoblots comparing 3 normal (N1-N3) and 3 asthmatic (A1-A3) HASM cell lines demonstrate that, under the same protein loading conditions yielding similar β-actin levels, constitutive expression of phosphorylated c-Src (p-Src) together with free Gβ protein are distinctly increased in the asthmatic HASM cell lines. Representative immunoblots in (FIG. 3C) and (FIG. 3D) demonstrate that, relative to normal cells, constitutively increased p-Src and p-ERK1/2 levels detected in asthmatic HASM cells, respectively, are acutely suppressed by treatment with either the anti-Gβγ blocking peptide or gallein (1 μM×30 min), similar to the suppressive effect of treatment with the c-Src-selective inhibitor, SU6656. (FIG. 3E) Treatment of normal HASM cells with IL-13 (50 ng/ml) acutely evokes temporal increases in free Gβ protein levels, peaking at 60-90 min and remaining elevated above baseline at 120 min. in free (activated). (FIG. 3F) Co-IP experiment demonstrating that, relative to unstimulated cells, IL-13-treated (50 ng/ml×60 min) normal HASM cells exhibit increased co-localization of p-Src with immunoprecipitated Gβ, and the latter is abrogated in IL-13-exposed HASM cells pretreated with either anti-Gβγ blocking peptide or gallein. The immunoblots shown in A-F are representative from 3-4 experiments.

FIG. 4. Comparison of effects of small molecule inhibitors of specific Gβγ-regulated downstream signaling proteins on PDE activity in asthmatic vs. normal HASM cells. Relative to baseline in normal HASM cells, constitutively heightened baseline PDE activity in asthmatic HASM cells is suppressed to near normal levels at 2 hr following treatment with either the c-Src inhibitor, SU6656, or the MEK-ERK1/2 inhibitor, U0126, whereas treatment with inhibitors of either p38 MAPK (SB202190), PI3K (LY294002), or PKA (H89) had no significant effect. Note: neither inhibitor had an appreciable effect on PDE activity in normal HASM cells. Data are mean±SE values, each based on 3-6 determinations. Comparisons between asthmatic vs. normal HASM cells are made using two-tailed Student t-test. *p<0.05; **p<0.01.

FIG. 5. Inhibition of Gβγ signaling suppresses intrinsically increased direct coupling of p-ERK1/2 and PDE4D proteins in asthmatic HASM cells. Western blotting in Co-IP analysis demonstrating that, under the same protein loading conditions yielding similar β-actin levels, relative to normal cells, asthmatic HASM cells exhibit distinctly increased direct co-localization of immunoblotted (IB) PDE4D with immunoprecipitated (IP) ERK1/2, the latter associated with heightened IB-detected p-ERK1/2. This heightened protein complex formation is acutely reversed (within 30 min) in asthmatic HASM cells treated with either anti-Gβγ blocking peptide or gallein. The immunoblots shown are representative from 3 experiments.

FIGS. 6A-6B. Constitutively increased co-localization of Rap1GAP with Gα protein contributes to upregulated PDE activity in asthmatic HASM cells. (FIG. 6A) Co-IP analysis demonstrating that, under comparable protein loading given by similar immunoblotted levels of co-immunoprecipitated Gα with anti-Gα immunoprecipitate, relative to 3 normal HASM cell lines, 3 asthmatic HASM cell lines exhibit markedly increased levels of co-immunoprecipitated Rap1GAP. (FIG. 6B) Relative to normal HASM cells, intrinsically heightened PDE activity in asthmatic HASM cells is significantly suppressed by ˜40% (p<0.05) in asthmatic cells transfected with a pool of siRNA duplexes targeted against Rap1GAP. By comparison, neither transfection with a scrambled (negative control) siRNA sequence (scRNA) nor pools of siRNA duplexes independently directed against RGS14 and PKAα had a significant effect on PDE activity in asthmatic HASM cells. Data are mean±SE values, each based on 3-5 determinations. Comparisons between asthmatic vs. normal HASM cells, and between scRNA-vs. Rap1GAP siRNA-transfected asthmatic HASM cells, are made using two-tailed Student t-test. *p<0.05; **p<0.01. Note: neither of the siRNA preparations had a significant effect on PDE activity in normal HASM cells (data not shown).

### DETAILED DESCRIPTION OF THE INVENTION

Signaling by the Gβγ subunit of Gi protein, leading to downstream c-Src-induced activation of the Ras/c-Raf1/MEK-ERK1/2 signaling pathway and its upregulation of phosphodiesterase-4 (PDE4) activity, was recently shown to mediate the heightened contractility in pro-asthmatic sensitized isolated airway smooth muscle (ASM), as well as allergen-induced airway hyper-responsiveness and inflammation in an in vivo animal model of allergic asthma. This study investigated whether cultured human ASM (HASM) cells derived from asthmatic donor lungs exhibit constitutively increased PDE activity that is attributed to intrinsically upregulated Gβγ signaling coupled to c-Src activation of the Ras/MEK/ERK1/2 cascade. We show that, relative to normal cells, asthmatic HASM cells exhibit markedly increased intrinsic PDE4 activity due to heightened Gβγ-regulated phosphorylation of c-Src and ERK1/2, and direct co-localization of the latter with the PDE4D isoform. These signaling events and their induction of heightened PDE activity are acutely suppressed by treating asthmatic HASM cells with a Gβγ inhibitor. Importantly, along with increased Gβγ activation, asthmatic HASM cells also exhibit constitutively increased direct binding of the small Rap1 GTPase-activating protein, Rap1GAP, to the α-subunit of Gi protein, which serves to cooperatively facilitate Ras activation and, thereby, enable enhanced Gβγ-regulated ERK1/2-stimulated PDE activity. Collectively, these data are the first to identify that intrinsically increased signaling via the Gβγ subunit, facilitated by Rap1GAP recruitment to the α-subunit, mediates the constitutively increased PDE4 activity detected in asthmatic HASM cells. These new findings support the notion that interventions targeted at suppressing Gβγ signaling both directly and secondary to inhibition of Rap1GAP activity may lead to novel approaches to treat asthma.

### I. Definitions

The following definitions are provided to facilitate an understanding of the present invention:

As used herein, the term “Rap1GAP inhibitor” refers to any molecule or compound which is able to disrupt Rap1GAP interactions and thereby impede downstream signaling. In certain embodiments, such inhibitors will be delivered in a targeted fashion, preferably to the airway. Such inhibitors have been previously described. See for example, Tsygankova O M, et al., J Biol Chem. 2013 Aug. 23; 288(34):24636-46. Another approach entails use of antisense nucleic acids which hybridize to the Rap1GAP encoding nucleic acid. Such antisense molecules are typically between 15-30 nucleotides in length but can also be complementary to the entire coding region.

By the term “asthmatic state” as used herein, is meant the proasthmatic phenotype which is observed in airway smooth muscle cells. This phenotype is characterized by increased contraction and decreased relaxation of the airway tissue when it has been exposed for extended time periods to cAMP-elevating agents such as beta2-adrenergic agonists, pro-asthmatic stimuli such as specific cytokines, high IgE-containing atopic asthmatic serum or exogenous IgE, compared with airway tissue which has not been exposed to these agents or stimuli. By the term “treating asthma” is meant curing asthma, causing the symptoms of asthma to diminish, ablating or otherwise alleviating the disease.

The term “aerosol formulation” refers to a pharmaceutical composition suitable for administration through the respiratory system or nasal passages. Examples of aerosol formulations are described below. Similarly, the term “aerosol administration” is intended to refer to a mode of administering an aerosol formulation to the respiratory system or nasal passages.

The term “substantially pure” refers to a preparation comprising at least 50-60% by weight of a given material (e.g., peptide, protein, etc.). More preferably, the preparation comprises at least 75% by weight, and most preferably 90-95% by weight of the given compound. Purity is measured by methods appropriate for the given compound (e.g. chromatographic methods, agarose or polyacrylamide gel electrophoresis, HPLC analysis, and the like).

“Inflammation-controlling effective amount” refers to the amount of the pharmaceutically active substance sufficient to elicit at least a desired threshold response to the substance in a subject to which the substance is administered, whether therapeutic or prophylactic.

The term “functional” as used herein implies that the nucleic or amino acid sequence is functional for the recited assay or purpose.

The phrase “consisting essentially of” when referring to a particular amino acid means a sequence having the properties of a given SEQ ID NO. For example, when used in reference to an amino acid sequence, the phrase includes the sequence per se and molecular modifications that would not affect the essential and novel characteristics of the sequence.

As disclosed herein, Rap1GAP inhibitors are effective at reducing a sign or symptom of asthma and thus are useful for the treatment thereof. The compositions of the invention are effective at inhibiting pro-asthmatic changes in airway smooth muscle tissue and may also be effective for treatment of allergic rhinitis, atopic dermatitis and possibly non-allergic rhinitis and dermatitis induced by chemical irritants.

### II. Pharmaceutical Compositions

Methods of the invention directed to treating asthma involve the administration of a Rap1GAP inhibitor in a pharmaceutical composition. A Rap1GAP inhibitor is administered to an individual as a pharmaceutical composition comprising a Rap1GAP inhibitor and a pharmaceutically acceptable carrier. Pharmaceutically acceptable carriers are well known in the art and include aqueous solutions such as physiologically buffered saline, other solvents or vehicles such as glycols, glycerol, oils such as olive oil or injectable organic esters.

A pharmaceutically acceptable carrier can contain physiologically acceptable compounds that act, for example, to stabilize the Rap1GAP inhibitor or increase the absorption of the agent. Such physiologically acceptable compounds include, for example, carbohydrates, such as glucose, sucrose or dextrans, antioxidants, such as ascorbic acid or glutathione, chelating agents, low molecular weight proteins or other stabilizers or excipients. One skilled in the art would know that the choice of a pharmaceutically acceptable carrier, including a physiologically acceptable compound, depends, for example, on the route of administration of the Rap1GAP inhibitor and on the particular physico-chemical characteristics of the peptide.

One skilled in the art appreciates that a pharmaceutical composition comprising a Rap1GAP inhibitor can be administered to a subject by various routes including, for example, orally or parenterally, such as intravenously (i.v.), intramuscularly, subcutaneously, intraorbitally, intranasally, intracapsularly, intraperitoneally (i.p.), intracistenally, intra-tracheally (i.t), or intra-articularly or by passive or facilitated absorption, and most preferably, using a nasal spray or inhalant.

Administration of a Rap1GAP inhibitor by inhalation is a particularly preferred means of treating an individual having asthma. One skilled in the art would recognize that a Rap1 gAP inhibitor can be suspended or dissolved in an appropriate pharmaceutically acceptable carrier and administered, for example, directly into the lungs using a nasal spray or inhalant.

A pharmaceutical composition comprising a Rap1GAP inhibitor can be administered as an aerosol formulation which contains the inhibitor in dissolved, suspended or emulsified form in a propellant or a mixture of solvent and propellant. The aerosolized formulation is then administered through the respiratory system or nasal passages.

An aerosol formulation used for nasal administration is generally an aqueous solution designed to be administered to the nasal passages in drops or sprays. Nasal solutions are generally prepared to be similar to nasal secretions and are generally isotonic and slightly buffered to maintain a pH of about 5.5 to about 6.5, although pH values outside of this range can additionally be used. Antimicrobial agents or preservatives can also be included in the formulation.

An aerosol formulation used for inhalations and inhalants is designed so that the Rap1GAP inhibitor is carried into the respiratory tree of the patient administered by the nasal or oral respiratory route. Inhalation solutions can be administered, for example, by a nebulizer. Inhalations or insufflations, comprising finely powdered or liquid drugs, are delivered to the respiratory system as a pharmaceutical aerosol of a solution or suspension of the drug in a propellant.

An aerosol formulation generally contains a propellant to aid in disbursement of the Rap1GAP inhibitor. Propellants can be liquefied gases, including halocarbons, for example, fluorocarbons such as fluorinated chlorinated hydrocarbons, hydrochlorofluorocarbons, and hydrochlorocarbons as well as hydrocarbons and hydrocarbon ethers (Remington's Pharmaceutical Sciences 18th ed., Gennaro, A. R., ed., Mack Publishing Company, Easton, Pa. (1990)).

Halocarbon propellants useful in the invention include fluorocarbon propellants in which all hydrogens are replaced with fluorine, hydrogen-containing fluorocarbon propellants, and hydrogen-containing chlorofluorocarbon propellants. Halocarbon propellants are described in Johnson, U.S. Pat. No. 5,376,359, and Purewal et al., U.S. Pat. No. 5,776,434.

Hydrocarbon propellants useful in the invention include, for example, propane, isobutane, n-butane, pentane, isopentane and neopentane. A blend of hydrocarbons can also be used as a propellant. Ether propellants include, for example, dimethyl ether as well as numerous other ethers.

The Rap1GAP inhibitor can also be dispensed with a compressed gas. The compressed gas is generally an inert gas such as carbon dioxide, nitrous oxide or nitrogen.

An aerosol formulation of the invention can also contain more than one propellant. For example, the aerosol formulation can contain more than one propellant from the same class such as two or more fluorocarbons. An aerosol formulation can also contain more than one propellant from different classes. An aerosol formulation can contain any combination of two or more propellants from different classes, for example, a fluorohydrocarbon and a hydrocarbon.

Effective aerosol formulations can also include other components, for example, ethanol, isopropanol, propylene glycol, as well as surfactants or other components such as oils and detergents (Remington's Pharmaceutical Sciences, 1990; Purewal et al., U.S. Pat. No. 5,776,434). These aerosol components can serve to stabilize the formulation and lubricate valve components.

The aerosol formulation can be packaged under pressure and can be formulated as an aerosol using solutions, suspensions, emulsions, powders and semisolid preparations. A solution aerosol consists of a solution of an active ingredient such as a Rap1GAP inhibitor in pure propellant or as a mixture of propellant and solvent. The solvent is used to dissolve the active ingredient and/or retard the evaporation of the propellant. Solvents useful in the invention include, for example, water, ethanol and glycols. A solution aerosol contains the active ingredient peptide and a propellant and can include any combination of solvents and preservatives or antioxidants.

An aerosol formulation can also be a dispersion or suspension. A suspension aerosol formulation will generally contain a suspension of an effective amount of the Rap1GAP inhibitor and a dispersing agent. Dispersing agents useful in the invention include, for example, sorbitan trioleate, oleyl alcohol, oleic acid, lecithin and corn oil. A suspension aerosol formulation can also include lubricants and other aerosol components.

An aerosol formulation can similarly be formulated as an emulsion. An emulsion can include, for example, an alcohol such as ethanol, a surfactant, water and propellant, as well as the active ingredient, Rap1GAP inhibitor. The surfactant can be nonionic, anionic or cationic. One example of an emulsion can include, for example, ethanol, surfactant, water and propellant. Another example of an emulsion can include, for example, vegetable oil, glyceryl monostearate and propane.

An aerosol formulation containing a Rap1GAP inhibitor will generally have a minimum of 90% of the particles in inhalation products between about 0.5 and about 10 μm to maximize delivery and deposition of the Rap1GAP inhibitor to respiratory fluids. In particular, the particle size can be from about 3 to about 6 μm.

A pharmaceutical composition comprising a Rap1GAP inhibitor also can be incorporated, if desired, into liposomes, microspheres, microbubbles, or other polymer matrices (Gregoriadis, Liposome Technology, Vols. I to III, 2nd ed., CRC Press, Boca Raton Fla. (1993)). Liposomes, for example, which consist of phospholipids or other lipids, are nontoxic, physiologically acceptable and metabolizable carriers that are relatively simple to make and administer.

In order to treat an individual having asthma, to alleviate a sign or symptom of the disease, a Rap1GAP inhibitor should be administered in an effective dose. The total treatment dose can be administered to a subject as a single dose or can be administered using a fractionated treatment protocol, in which multiple doses are administered over a more prolonged period of time, for example, over the period of a day to allow administration of a daily dosage or over a longer period of time to administer a dose over a desired period of time. One skilled in the art would know that the amount of a Rap1GAP inhibitor required to obtain an effective dose in a subject depends on many factors, including the age, weight and general health of the subject, as well as the route of administration and the number of treatments to be administered. In view of these factors, the skilled artisan would adjust the particular dose so as to obtain an effective dose for treating an individual having asthma.

The effective dose of a Rap1GAP inhibitor will depend on the mode of administration, and the weight of the individual being treated. The dosages described herein are generally those for an average adult but can be adjusted for the treatment of children. The dose will generally range from about 0.001 mg to about 1000 mg.

The concentration of a Rap1GAP inhibitor in a particular formulation will depend on the mode and frequency of administration. A given daily dosage can be administered in a single dose or in multiple doses so long as the Rap1GAP inhibitor concentration in the formulation results in the desired daily dosage. One skilled in the art can adjust the amount of Rap1GAP inhibitor in the formulation to allow administration of a single dose or in multiple doses that provide the desired concentration of Rap1GAP inhibitor over a given period of time.

In an individual suffering from asthma, in particular a more severe form of the disease, administration of a Rap1GAP inhibitor can be particularly useful when administered in combination, for example, with a conventional agent for treating such a disease. The skilled artisan would administer a Rap1GAP inhibitor, alone or in combination with a second agent, based on the clinical signs and symptoms exhibited by the individual and would monitor the effectiveness of such treatment using routine methods such as pulmonary function determination, radiologic, immunologic or, where indicated, histopathologic methods.

A Rap1GAP inhibitor can be administered in combination with steroidal anti-inflammatory agents including corticosteroids, for example, dexamethasone, beclomethasone, fluticasone, triamcinolone and budesonide. A Rap1GAP inhibitor can also be administered in combination with non-steroidal anti-inflammatory agents such as, indomethacin, ibuprofen, naproxen, diclofenac, sulindac, oxaprozin, diflunisal, bromfenac, piroxicam, etodolac and fenoprofen Inhibitor administration can also be combined with short- and long-acting β2-adrenergic agents such as albuterol and salmeterol, respectively, as the inhibitor alleviates the desensitization to the β2-adrenoreceptor agent. When a Rap1GAP inhibitor is used with another anti-inflammatory agent, the Rap1GAP inhibitor can generally be administered at a lower dosage. For example, a Rap1GAP inhibitor can be administered at a dose of less than 10 mg per day in combination with another anti-inflammatory agent.

When a Rap1GAP inhibitor is administered in combination with one or more other anti-inflammatory agent, the Rap1GAP inhibitor and other anti-inflammatory agent can be co-administered in the same formulation. Alternatively, the Rap1GAP inhibitor and other anti-inflammatory agent can be administered simultaneously in separate formulations. In addition, the Rap1GAP inhibitor can be administered in separate formulations, where the separate formulations are not administered simultaneously but are administered during the same period of treatment, for example, during a daily or weekly period of treatment. Alternatively, each agent may be given sequentially during a daily or weekly period of treatment.

Administration of the pharmaceutical preparation is preferably in a “prophylactically effective amount” or a “therapeutically effective amount” (as the case may be, although prophylaxis may be considered therapy), this being sufficient to show benefit to the individual. This amount prevents, alleviates, abates, or otherwise reduces the severity of symptoms in a patient.

The pharmaceutical preparation is formulated in dosage unit form for ease of administration and uniformity of dosage. Dosage unit form, as used herein, refers to a physically discrete unit of the pharmaceutical preparation appropriate for the patient undergoing treatment. Each dosage should contain a quantity of active ingredient calculated to produce the desired effect in association with the selected pharmaceutical carrier. Procedures for determining the appropriate dosage unit are well known to those skilled in the art.

Dosage units may be proportionately increased or decreased based on the weight of the patient. Appropriate concentrations for alleviation of a particular pathological condition may be determined by dosage concentration curve calculations, as known in the art. As mentioned previously, a preferred embodiment of the invention comprises aerosolized delivery of the Rap1GAP inhibitor to the lungs of a patient in need thereof. The Rap1GAP inhibitor described herein can also be injected intra-peritoneally (i.p.), intravenously (i.v.), or intratracheally (i.t.). Formulation, dosages and treatment schedules have also been described hereinabove.

The following materials and methods are provided to facilitate the practice of the present invention.

Materials:

All chemicals were purchased from Sigma-Aldrich unless otherwise indicated.

Culture and Treatment of HASM Cells:

Primary cultures of HASM cells derived from bronchial smooth muscle freshly isolated post-mortem from normal non-asthmatic (n=3) and asthmatic (n=4) donor lungs were purchased at passage 2-3 from Lonza Walkersville, Inc. Other than data regarding age, race, and gender of the donors provided in Table 1, limited information was available with respect to clinical severity of asthma, treatment history, or cause of death, with exception to asthma donor #1. All the experiments were conducted at passage 5-6, after growing the cells in Lonza's serum-containing SmBM medium supplemented with the BulletKit medium that contains insulin, human FGF, gentamycin/amphotericin B. The cells were maintained throughout in a humidified incubator containing 5% CO₂ in air at 37° C. and, after attaining ˜90% confluence, cells were growth arrested for 24 hr in serum-free Ham's F12 medium. Thereafter, the cells were examined for constitutive and induced changes in PDE activity, levels of free Gβ, c-Src and ERK1/2 phosphorylation, and PDE4D protein expression, all in the absence and presence of different pretreatment conditions, as described.

Assay of cAMP PDE Activity:

Levels of total cAMP PDE activity were determined in normal and asthmatic HASM cells at baseline and at different times ranging from 0.25-24 hr following administration of a maximally effective concentration of either: 1) a cell permeable anti-Gβγ blocking peptide (1 μM; AnaSpec; Premont, Calif.), comprised of a membrane permeable sequence (MPS; 15 amino acids) conjugated to the Gβγ-sequestering C-terminal domain (28 amino acids) of phosducin-like protein [28]; 2) the inert MPS peptide alone (1 μM) serving as a negative-control; or 3) 1 μM of gallein [29], a recently described small molecule inhibitor of Gβγ signaling [30] (Acros Organics). PDE activity was also assayed in normal and asthmatic HASM cells following dose-dependent administration of anti-Gβγ blocking peptide (0.01-10 μM), and following treatment with various small molecule inhibitors, as described. The PDE activity assay was performed in the HASM cell lysates using a colorimetric, non-radioactive enzymatic assay (Enzo Life Sciences; Plymouth Meeting, Pa.) as per the manufacturer's protocol, with some modification of our previously described approach [16,17]. Specifically, to maximize the sensitivity and yield of the measurement enzyme activity, we used materials including a cell lysis buffer completely free of phosphate, composed of 50 mM Tris, pH 7.5, 0.1 mM EDTA, 0.1 mM EGTA, 1 mM DTT and 0.2% NP-40. The measured levels of PDE activity were standardized to protein content in the cell extracts. Finally, PDE activity was also assayed in normal and asthmatic HASM cells following transfection with siRNA preparations targeted at suppressing specific signaling molecules, as described below.

siRNA-Mediated Knockdown Studies:

Targeted protein suppression using siRNA preparations was examined in normal and asthmatic HASM cells that were initially seeded into 6-well plates and, at ˜40% confluency, the medium was replaced with the reduced serum-containing medium, Opti-MEM (Invitrogen). Cells were then transfected twice during a 24-hr interval with pools of siRNA preparations, each pool comprised of three siRNA duplexes targeted against either human Rap1GAP (Ambion; ID: s11785, s11786, s11787), RGS14, (Ambion; ID: 18059, 18147, 135857), or the PKAα catalytic subunit (Santa Cruz Biotechnology; Pool ID: sc-36240), as well as a non-targeted (scrambled) siRNA duplex serving as a negative control. As previously described [16.17.31], the transfections were carried out using Oligofectamine (Invitrogen) as the transfection agent, and the pools of siRNAs were applied to each well at a final concentration of 100 nM for each siRNA preparation. Consistent with our previous reports [16,17,31], this double-transfection approach enabled high transfection efficiency and, as detected by Western blotting, markedly inhibited expression of the targeted proteins by their respective siRNA preparations, with maximal inhibition detected at 72 hr following siRNA transfection averaging 82, 89, and 92% for RGS14, Rap1GAP and PKA proteins, respectively.

Immunoblot Analysis of Gβγ-Regulated c-Src and ERK1/2 Phosphorylation.

Levels of total and phosphorylated c-Src at residue Tyr416 and ERK1/2 proteins, as well as Gβ protein, were detected by Western blot analysis of lysates isolated from normal and asthmatic HASM cells before and at various times after treatment either with 1 μM of anti-Gβγ blocking peptide or gallein, or the c-Src family tyrosine kinase-selective inhibitor, SU6656 (10 μM), or exposure to the pro-asthmatic Th2 cytokine, IL-13 (50 ng/ml), as described. Following protein extraction and addition of gel loading buffer, the cellular extracts were loaded on a 10% SDS-PAGE gel for immunoblotting after transfer to a PVDF membrane. The membranes were then incubated overnight with monoclonal mouse anti-human primary antibodies directed against c-Src, phospho-c-Src^{Tyr416}, ERK1/2 and phospho-ERK1/2 (Millipore; Billerica, Mass.), and a rabbit anti-human polyclonal antibody directed against the Gβ subunit (Millipore; Billerica, Mass.). Protein levels were detected by ECL after a 1-hr incubation with a 1:2,000 dilution of HRP-conjugated secondary antibody, followed by exposure to autoradiography film. The protein band intensities were quantified by densitometry.

Co-Immunoprecipitation Studies.

Normal and asthmatic HASM cells were prepared for co-immunoprecipitation (Co-IP) studies, performed as previously described [18], under native conditions in order to preserve protein-protein associations. After indicated treatments, cells were harvested and then exposed to lysis buffer. 1.5 mg of Protein G Dynabeads (Invitrogen; Carlsbad, Calif.) were incubated with 10 μg of anti-Gβ rabbit IgG (Millipore) or anti-Gα (pan) rabbit polyclonal antibody (Millipore; Billerica, Mass.) and incubated for 10 min at room temperature with rotation. Following several washes, the bound bead/antibody complex was added to sample, mixed by pipetting, and incubated for 2 hr at 4° C. with rotation. The captured bead/Protein G/antigen complex was then washed several times and eluted at pH 3.0 with rotation at room temperature. The precipitated immunocomplexes were subsequently analyzed by immunoblotting using either anti-Gβ, anti-phospho-c-Src^{Tyr416}, anti-ERK1/2, or anti-PDE4D rabbit polyclonal antibody, or anti-Rap1GAP rabbit monoclonal IgG antibody (Millipore; Billerica, Mass.).

Statistical Analyses.

Results are expressed as mean±SE values. Comparisons between normal and asthmatic HASM cell treatment groups were made using the Student t test (2-tailed). A probability of <0.05 was considered statistically significant. Statistical analyses were conducted by using the Prism computer program by Graph Pad Software Inc.

The following example is provided to illustrate certain embodiments of the invention. It is not intended to limit the invention in any way.

### Example

In light of recent studies demonstrating a pivotal role for PDE4 activity in regulating airway function in asthmatic individuals [19-21] and in animal models of allergic asthma [22-26], and that PDE4 activity is intrinsically increased in cultured human ASM (HASM) cells isolated from asthmatic individuals [27], the present study sought to determine whether asthmatic HASM cells exhibit constitutively increased PDE activity that is mechanistically attributed to intrinsically upregulated Gβγ signaling coupled to c-Src-induced activation of the Ras/MEK/ERK1/2 pathway. The results demonstrated that: 1) relative to normal (non-asthmatic) HASM cells, primary cultures of asthmatic HASM cells exhibit markedly increased constitutive PDE4 activity associated with free (activated) Gβγ-coupled c-Src and ERK1/2 activation; 2) this Gβγ-regulated increase in PDE activity is attributed to intrinsically enhanced co-localization of phosphorylated ERK1/2 with the PDE isoform, PDE4D, and 3) inhibition of Gβγ signaling acutely suppresses (within minutes) the increased PDE activity in asthmatic HASM cells to near normal levels, in association with suppression of c-Src and ERK1/2 activation, and co-localization of the latter with PDE4D. Finally, together with increased PDE activity attributed to free Gβγ-regulated ERK1/2 activation, the results demonstrated that asthmatic HASM cells also exhibit markedly increased direct binding of the small Rap1 GTPase-activating protein (Rap1GAP) to the α-subunit of G protein, a phenomenon that serves to cooperatively facilitate Ras-induced ERK1/2 activation, thereby enabling enhanced Gβγ-regulated PDE activity. Taken together, these new findings are the first to identify that asthmatic HASM cells exhibit constitutively increased PDE activity that is mechanistically attributed to intrinsically increased Gβγ signaling, facilitated by Rap1GAP recruitment to the Gα-subunit, leading to heightened c-Src-dependent/Ras-mediated activation of ERK1/2 and its consequent direct binding to and activation of PDE4. Given the crucial role attributed to upregulated PDE activity in the pathobiology of asthma, these new findings highlight a heretofore-unidentified decisive role for Gβγ signaling in regulating the heightened PDE4 activity that characterizes the asthmatic airway, and suggest that interventions targeted at suppressing Gβγ signaling associated with Gi protein activation may lead to novel approaches to treat asthma.

Intrinsically Increased PDE Activity in Asthmatic HASM Cells is Mediated by Activated Gβγ Signaling.

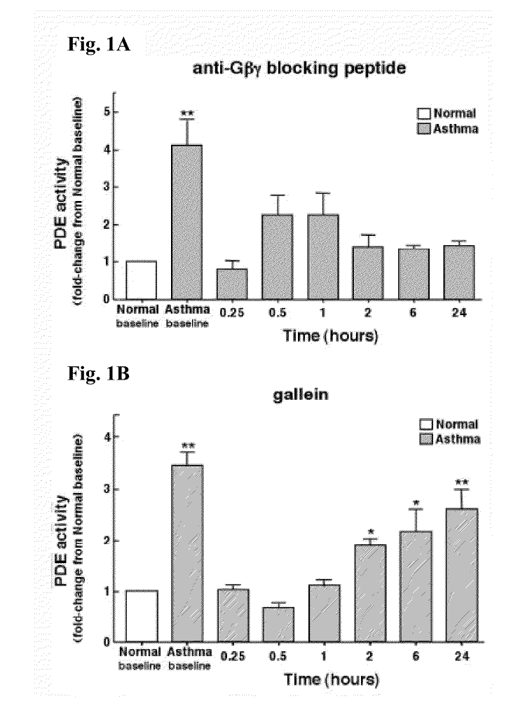

We previously implicated upregulated PDE4 activity due to activation of the βγ subunit of Gi protein in mediating airway constrictor hyperresponsiveness and inflammation in an in vivo rabbit model of allergic asthma [18], as well as the altered contractility elicited in isolated ASM tissues sensitized under various atopic-related and -unrelated proasthmatic conditions [16-18,32]. Given this evidence, together with that demonstrating a critical role for increased PDE4 activity in regulating airway function in asthmatic individuals [19-21], and that PDE4 activity is intrinsically increased in cultured asthmatic HASM cells [27], we hypothesized that asthmatic HASM cells exhibit constitutively increased PDE4 activity that is mechanistically attributed to upregulated Gβγ signaling. Initial studies demonstrated that, relative to normal (non-asthmatic) HASM cells wherein PDE activity averaged 6.25±0.91 nmol/min/mg protein, basal PDE activity was markedly increased by approximately four-fold in cultures of asthmatic HASM cells (FIG. 1A). Treatment of the asthmatic cells with a maximally effective concentration of the anti-Gβγ blocking (sequestering) peptide (1 μM), comprised of the C-terminal domain of PhLP conjugated to an inert membrane permeable carrier peptide (MPS) [28], acutely suppressed PDE activity within 15 min to levels comparable to those detected in normal HASM cells, and this suppression was largely sustained for up to 24 hr (FIG. 1A). PDE activity was similarly acutely suppressed in asthmatic HASM cells that were treated with gallein (1 μM), a small molecule inhibitor of Gβγ signaling [29,30] (FIG. 1B). Unlike the anti-Gβγ blocking peptide, however, the suppressive effect of gallein was transient in nature, with progressive recovery in PDE activity that approached the heightened basal levels by 24 hr.

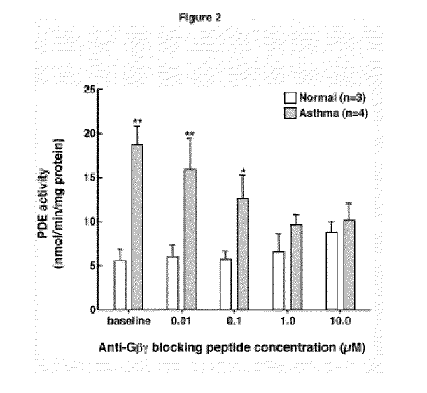

Because of its sustained action, the dose-dependent effects of separate treatments (each lasting 6 hr) with increasing concentrations of the anti-Gβγ blocking peptide on PDE activity were next assessed in HASM cells isolated from 3 normal and 4 asthmatic individuals. Unlike in normal cells wherein PDE activity remained largely unaltered, asthmatic HASM cells exhibited dose-dependent suppression of PDE activity following administration of the anti-Gβγ blocking peptide (FIG. 2). Maximal inhibition of PDE activity to near normal levels was attained using 1 μM of the anti-Gβγ peptide, and no further suppressive effect was detected following administration of 10 μM. In relation to the these observations, it should be noted that results obtained in separate experiments demonstrated that the heightened constitutive PDE activity in asthmatic HASM cells was also completely abrogated following treatment with the selective PDE4 inhibitor, rolipram (10 μM×1 hr; data not shown), a finding consistent with our previously reported observations demonstrating that rolipram inhibits the increased PDE activity evoked in cultured normal HASM cells and isolated rabbit ASM tissues sensitized under various proasthmatic conditions [16-18]. Moreover, this finding concurs with the observations in a recent report demonstrating that the increased PDE activity detected in cultured asthmatic HASM cells is inhibited by treatment with other selective PDE4 inhibitors, including roflumilast and cilomilast [27]. Thus, the above results demonstrate that the intrinsically increased PDE activity exhibited in asthmatic HASM cells is attributed to Gβγ-regulated activation of the PDE4 subtype (see below).

Gβγ-Regulated c-Src-Coupled ERK1/2 Activation is Intrinsically Increased in Asthmatic HASM Cells.

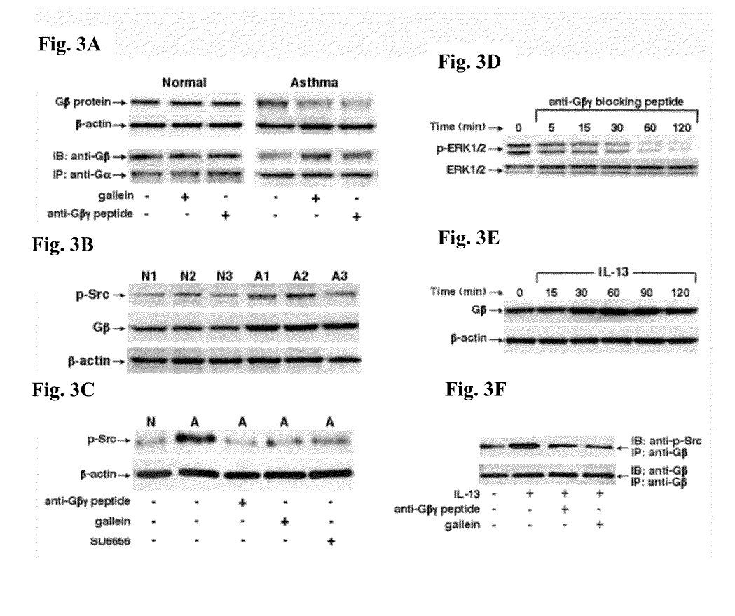

In light of above results, a series of studies were conducted to systematically examine whether the intrinsically heightened Gβγ-regulated PDE activity in asthmatic HASM cells is mechanistically related to constitutively increased Gβγ activation associated with c-Src-induced ERK1/2 activation and its consequent acute stimulation of PDE activity. Accordingly, initial co-immunoprecipitation experiments compared intracellular levels of membrane-bound Gα-associated and -dissociated (“free”) Gβ, reflecting the inactive and activated states of G protein signaling, respectively, as well as levels of activated (phosphorylated) c-Src and ERK1/2 in lysates isolated from normal and asthmatic HASM cells under different treatment conditions. The representative immunoblots in FIG. 3A demonstrate that, under comparable protein loading conditions yielding similar β-actin levels, relative to normal cells, untreated asthmatic HASM cells exhibited constitutively increased free Gβ levels and, correspondingly, reduced co-localization of Gβ with immunoprecipitated Gα. Furthermore, this pattern of Gβ distribution, reflective of constitutively heightened Gβγ activation, was acutely reversed in asthmatic HASM cells that were treated either with gallein or the anti-Gβγ blocking peptide (1 μM), as evidenced by increased Gα-associated and, correspondingly, reduced free Gβ levels detected at 30 min following treatment with either Gβγ inhibitor, whereas neither inhibitor had an effect in normal cells (FIG. 3A).

To ascertain whether the heightened state of constitutive Gβγ activation in asthmatic HASM cells is associated with altered downstream signaling events, we next compared the levels of c-Src and ERK1/2 activation in asthmatic vs. normal HASM cells, both in the absence and presence of Gβγ inhibition. Activated c-Src, denoted by its state of autophosphorylation at residue Tyr416 [33], and activated (phosphorylated) ERK1/2 were assessed using phospho-c-SrcTyr416- and phospho-ERK1/2-specific antibodies, respectively. Immunoblot analysis of these phosphorylated proteins in HASM cell lines isolated from 3 normal and 3 asthmatic individuals demonstrated that, under the same loading conditions yielding similar β-actin protein levels, relative to the normal (N) cells, the increased free Gβ protein levels detected in asthmatic (A) HASM cells were associated with increased levels of phosphorylated c-SrcTyr416 (p-Src), whereas little p-Src was detected in normal cells (FIG. 3B). Moreover, in concert with its acute suppression of PDE activity, Gβγ inhibition with either the anti-Gβγ blocking peptide or gallein acutely suppressed both the constitutively increased levels of: 1) p-Src detected in asthmatic HASM cells, similar to the suppressive effect of treatment with the c-Src family tyrosine kinase-selective inhibitor, SU6656 (FIG. 3C); and 2) phosphorylated ERK1/2 (p-ERK1/2), in accordance with the concept that the intrinsically increased Gβγ-regulated phosphorylation of c-Src is coupled to downstream activation of the Ras/c-Raf1/MEK-ERK1/2 pathway in asthmatic HASM cells (FIG. 3D).

Finally, to determine whether the constitutively heightened state of Gβγ-regulated signaling in asthmatic HASM cells parallels that which may be acutely induced by exogenous pro-asthmatic stimulation, naive normal HASM cells were exposed for varying durations to the pro-asthmatic Th2 cytokine, IL-13 (50 μg/ml), and then examined for induced activation of Gβγ and its direct coupling to phosphorylated c-Src. The immunoblot in FIG. 3E demonstrates that treatment with IL-13 acutely evoked temporal increases in free Gβ levels that peaked at 60-90 min and declined thereafter, but remained elevated above baseline at 120 min. Moreover, as depicted by a representative co-immunoprecipitation experiment in FIG. 3F, relative to unstimulated cells, enhanced co-localization of p-Src with immunoprecipitated Gβ was detected in HASM cells treated for 60 min with IL-13, and this induced association was abrogated in IL-13-exposed HASM cells that were pretreated with either the anti-Gβγ blocking peptide or gallein. It should be noted that these data were obtained under similar conditions of loading of immunoprecipitated Gβ, as demonstrated by the corresponding immunoblots using anti-Gβ antibody. Thus, these results support the concept that IL-13 acutely induces Gβγ activation which is accompanied by direct coupling of activated Gβγ with c-Src, a signaling event previously associated with acute ERK1/2 activation in IL-13-exposed HASM cells [18].

Increased Gβγ-Regulated c-Src-Coupled ERK1/2 Activation Mediates Intrinsically Heightened PDE Activity in Asthmatic HASM Cells.

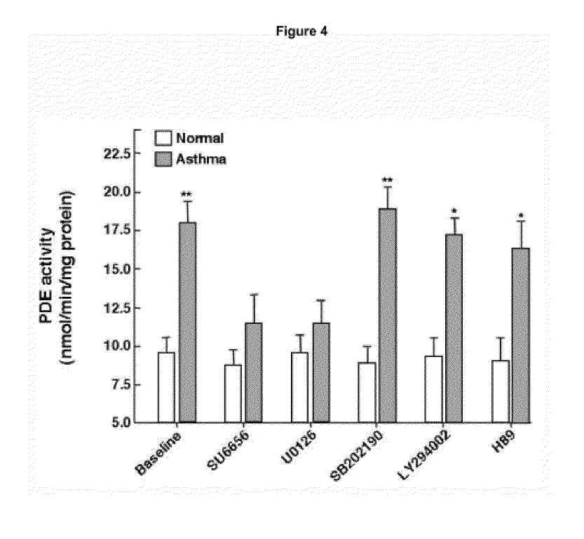

Given the above results, we next investigated the role of the aforementioned Gγγ-regulated/Src-coupled ERK1/2 signaling mechanism, as well as other potential downstream molecules, in mediating the increased PDE activity detected in asthmatic HASM cells. Accordingly, asthmatic and normal HASM cell cultures were compared with respect to changes in PDE activity induced by treatment with previously reported maximally effective concentrations of specific inhibitors of signaling molecules potentially associated with Gβγ activation and upregulated PDE4 activity. As shown in FIG. 4, the significantly heightened basal PDE activity exhibited by untreated asthmatic HASM cells was acutely reversed to near normal levels following exposure (×2 hr) to maximally effective concentrations of either the c-Src inhibitor, SU6656 (10 μM) or the MEK-ERK1/2 inhibitor, U0126 (5 μM), whereas treatment with inhibitors of either p38 MAPK (SB202190; 10 μM), PI3K (LY294002; 10 μM), or PKA (H89; 10 μM) had no significant effect. These data are consistent with the notion that the constitutively increased PDE activity in asthmatic HASM cells is largely attributed to a heightened state of Gβγ-regulated downstream signaling that involves increased c-Src-coupled ERK1/2 activation, rather than activation of the p38 MAPK, PI3K or PKA pathways.

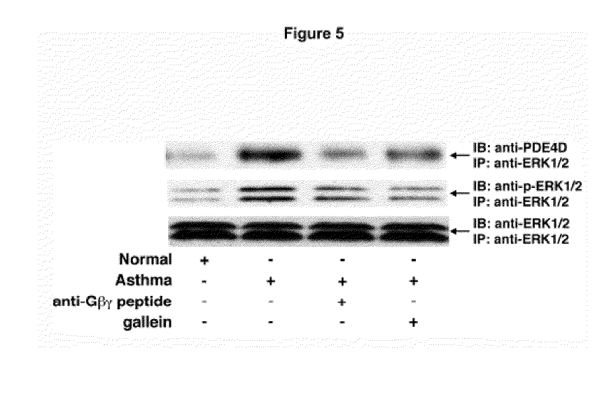

The above collection of data demonstrating that Gβγ inhibition can acutely (within minutes) reverse the increased PDE activity in asthmatic HASM cells raised the possibility that the enhanced PDE activity reflects a constitutively heightened state of Gβγ-regulated direct interaction between activated ERK1/2 and PDE4D, which represents the functionally relevant PDE4 isoform in HASM cells (34). Indeed, such a direct interaction involving ERK1/2-induced phosphorylation and activation of PDE4D has been previously demonstrated accompanying protein kinase C stimulation in vascular smooth muscle cells [35]. Co-immunoprecipitation experiments addressing this possibility herein demonstrated that, under comparable protein loading conditions yielding similar immunoblotted levels of co-immunoprecipitated total ERK1/2, relative to normal cells, asthmatic HASM cells exhibited increased levels of co-immunoprecipitated p-ERK1/2, as well as increased co-immunoprecipitation of PDE4D with the ERK1/2 immunoprecipitate, whereas minimal or undetectable levels of such PDE4D co-localization was observed in normal cells (FIG. 5). Moreover, the heightened state of co-localization of ERK1/2 with PDE4D was acutely reversed (within 30 min) following treatment of asthmatic HASM cells with either the anti-Gβγ blocking peptide or gallein (FIG. 5). Thus, these data together with the above observations demonstrate that the constitutively increased Gβγ-regulated/ERK1/2-mediated PDE activity exhibited in asthmatic HASM cells (FIGS. 3 and 4) reflects an intrinsically heightened state of direct interaction between activated ERK1/2 and PDE4D.

Role of the GTPase-Activating Protein, Rap1GAP, in Regulating PDE Activity in Asthmatic HASM Cells

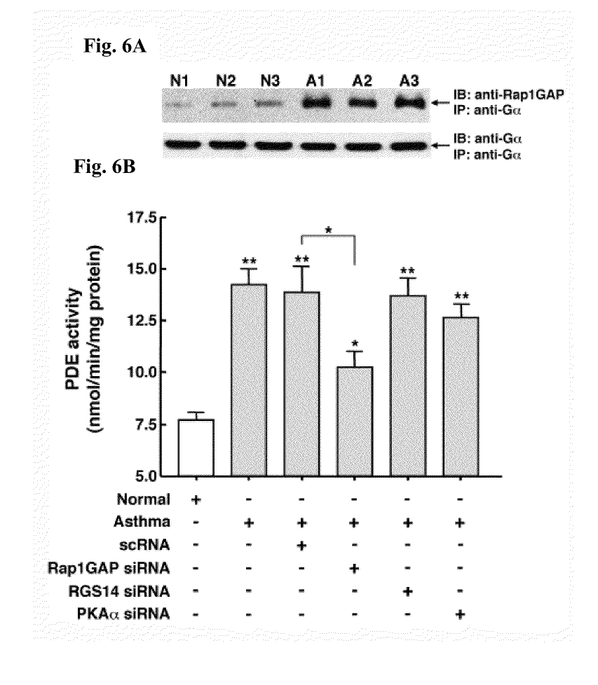

Ras-induced downstream activation of ERK1/2 is inhibited by the Ras-related small GTP-binding protein, Rap1, suggesting that the latter may serve as an intrinsic homeostatic inhibitor of ERK1/2 activation [36-38]. Rap1 activity itself, however, is subject to suppression by the Rap1 GTPase, Rap1GAP (also Rap1GAPII), a phenomenon that arguably serves to facilitate Ras-induced downstream ERK1/2 activation [36-41]. In light of this evidence, we sought to determine the potential role of Rap1GAP in modulating the above-identified Gβγ-regulated signaling events implicated in mediating the intrinsically increased PDE activity in asthmatic HASM cells. In this regard, it is noteworthy that, apart from acting as a GTPase activating protein (GAP), Rap1GAP also serves as a guanine nucleotide dissociation inhibitor (GDI) that binds via its GoLoco amino acid motif to the α-subunit of the Gi family of heterotrimeric G-proteins [40,41]. Initial co-immunoprecipitation experiments demonstrated that asthmatic HASM cells exhibit constitutively increased co-localization of Rap1GAP with Gα. As shown in FIG. 6A, in comparing 3 normal and 3 asthmatic cells lines under similar protein loading conditions, evidenced by comparable immunoblotted levels of co-immunoprecipitated Gα with anti-Gα immunoprecipitate, the asthmatic HASM cells exhibited strikingly increased levels of co-immunoprecipitated Rap1GAP, whereas minimal such co-localization was detected in the normal cells. Given these observations, the potential role of Rap1GAP in regulating PDE activity was next examined by comparing the levels of PDE activity in cells transfected over 48 hr with pools of siRNA duplexes directed against either Rap1GAP, RGS14 (another GAP/GDI protein that also binds Gαi via its GoLoco motif [42]), the catalytic α-subunit of protein kinase A (PKAα), or a scrambled siRNA (scRNA) sequence serving as a negative control. As shown in FIG. 6B, relative to normal cells, similar levels of significantly heightened PDE activity were detected in non-transfected and scRNA-transfected asthmatic HASM cells. This heightened activity was significantly suppressed by ˜40% in asthmatic HASM cells that were transfected with the Rap1GAP siRNA duplexes, although the mean level of PDE activity expressed in these cells remained significantly greater than that detected in normal cells. By comparison, transfection with siRNAs directed against either RGS14 or PKAα had no significant effect on PDE activity in asthmatic HASM cells. Finally, it should be noted that neither of the siRNA preparations significantly affected PDE activity in normal HASM cells (data not shown). Collectively, these results are consistent with the notion that the heightened PDE activity detected in asthmatic HASM cells is partly, but significantly, attributed to constitutively increased co-localization of Rap1GAP with Gα, a phenomenon that is ostensibly permissive of Gi-βγ-stimulated Ras-induced ERK1/2 activation consequent to inactivation of Rap1 by Rap1GAP. In this context, our data suggest that the latter mechanism involving Rap1 suppression due to Rap1GAP binding to Gαi acts cooperatively with that involving activation of Ras through Gβγ signaling to transduce heightened activation of the Ras-stimulated c-Raf-MEK-ERK cascade mediating increased PDE activity in asthmatic HASM cells.

### Discussion

Increased PDE4 activity has been shown to play a decisive role in mediating the airway constrictor hyper-responsiveness evoked by allergen challenge in asthmatic individuals [19,21] and in animal models of allergic asthma [18,22-26], as well as the pro-asthmatic changes in contractility in isolated ASM tissue that accompanies its prolonged β2AR desensitization [16,17] or passive sensitization with atopic asthmatic serum or IL-13 [18]. The upregulated PDE4 activity was attributed to a mechanism that involves PTX-sensitive Gi-βγ activation under the different sensitizing conditions, which triggers c-Src-induced stimulation of the Ras/c-Raf/MEK signaling pathway leading to ERK1/2-dependent transcriptional upregulation of the PDE4D5 isotype and its consequent induction of the proasthmatic phenotype in the sensitized ASM [16-18,32]. In light of this evidence, the present study sought to determine whether asthmatic HASM cells exhibit constitutively increased PDE activity that is due to intrinsically upregulated signaling via the aforementioned Gβγ-regulated mechanism. The results are the first to demonstrate that: 1) relative to normal HASM cells, cultured asthmatic HASM cells intrinsically exhibit markedly increased rolipram-sensitive PDE activity that is regulated by constitutively increased free (activated) Gβγ-induced c-Src and ERK1/2 activation; 2) this heightened Gβγ-regulated ERK1/2-induced PDE activity involves enhanced direct co-localization of activated ERK1/2 with the PDE4D isoform in the asthmatic HASM cells; and 3) inhibition of this intrinsically heightened Gβγ-regulated signaling mechanism acutely suppresses PDE activity in asthmatic HASM cells to near normal levels, in association with suppression of c-Src and ERK1/2 activation and reversal of co-localization of ERK1/2 with PDE4D. Finally, together with intrinsically increased PDE activity attributed to free Gβγ-regulated ERK1/2 activation, the results demonstrated that asthmatic HASM cells also exhibit constitutively increased co-localization of Rap1GAP with the G protein α-subunit, a cooperative phenomenon that serves to facilitate Gβγ-regulated ERK1/2-induced activation of PDE activity in the asthmatic HASM cells (see below). Thus, the present data identify that asthmatic HASM cells exhibit constitutively enhanced PDE4 activity that is regulated by increased Gβγ signaling which, together with membrane recruitment of Rap1GAP, evokes heightened c-Src-stimulated downstream activation of ERK1/2 and its direct coupling to PDE4D. Given the important role attributed to increased PDE activity in the pathobiology of asthma, these new findings highlight a heretofore-unknown mechanism whereby constitutively increased Gβγ signaling regulates the altered phenotype in asthmatic HASM cells, suggesting that interventions targeted at suppressing this Gβγ-regulated signaling mechanism may enable novel approaches to treat asthna.

Among various issues raised by the present observations, the finding that asthmatic HASM cells exhibit constitutively increased PDE activity was not unexpected, given recent independent evidence demonstrating intrinsically heightened PDE4 activity in cultured asthmatic vs. normal HASM cells [27], and that treatment of asthmatic individuals with the PDE4 inhibitor, roflumilast, decreases airway hyperresponsiveness following allergen challenge [19,21] and improves lung function [20]. The observation that treatment with a Gβγ inhibitor acutely suppresses (within 15 min) this increased PDE activity (FIG. 1), however, was not anticipated, as we had previously demonstrated that the heightened PDE activity evoked in cultured normal HASM cells and naive isolated rabbit ASM tissues by prolonged exposure (i.e., ˜24 hr) to various pro-asthmatic sensitizing conditions was due to transcriptional upregulation of PDE4D elicited by Gβγ-regulated ERK1/2 activation [16-18,32]. While such induced transcriptional control may also contribute to the increased PDE activity detected in asthmatic HASM cells, the observation that Gβγ inhibition acutely reverses this intrinsically increased PDE activity strongly suggests that another Gβγ-regulated signaling event(s) predominates in actively preserving the state of heightened PDE activity in asthmatic HASM cells, despite their maintenance in cell culture independent of any discernable persistent proinflammatory stimulation. This notion is supported by a series of present observations which demonstrated that, unlike in normal cells, asthmatic HASM cells exhibit constitutively increased Gβγ activity that acutely regulates Src and ERK1/2 activation (FIG. 3) which, in turn, mediates increased PDE activity (FIG. 4) accompanying direct coupling of activated ERK1/2 with PDE4D (FIG. 5). The latter finding concurs with those in an earlier study wherein, using comparably brief (<30 min) treatment regiments to exclude de novo protein synthesis, protein kinase C-induced stimulation of the Ras-c-Raf-MEK-ERK1/2 cascade was found to directly activate and translocate the membraneous PDE4D3 isoform in vascular smooth muscle cells [35]. On the other hand, contrasting this scenario, ERK1/2 activation was found to directly inhibit PDE4D5 activity in aortic smooth muscle cells, whereas co-activation of PKA reportedly “reprograms” ERK from causing such inhibition to net activation of PDE4D5 (43). In considering these discordant reported findings, it should be noted that our present observations demonstrated that, unlike treatment with the ERK1/2 inhibitor, U0126, which acutely suppressed the elevated PDE activity in asthmatic HASM cells to near normal levels, neither treatment with the PKA inhibitor, H89 (FIG. 4), nor transfection with a previously reported effective pool of PKA-directed siRNA preparations [16] had an appreciable effect (FIG. 6). Thus, these data together with those demonstrating that asthmatic HASM cells exhibit intrinsically increased co-localization of activated ERK1/2 with PDE4D, and that this co-localization is acutely reversed by treatment with a Gβγ inhibitor (FIG. 5), clearly implicate Gβγ-regulated ERK1/2 activation as playing a predominant role in directly inducing the increased PDE activity exhibited in asthmatic HASM cells. This evidence notwithstanding, a potential role for PKA activation and its modulation of ERK1/2-coupled regulation of PDE4 activity in asthmatic HASM cells cannot be completely ruled out.

While our present results are consistent with the prevailing concept that GPCR-dependent and receptor-independent stimulation of Ras-mediated ERK1/2 activation uses proximal signals generated by Gβγ-coupled c-Src activation [44-48], our data also concur with established evidence demonstrating that: 1) ERK1/2 activation is suppressed by recruitment of the GTP-binding protein, Rap1, which inhibits Ras function (36-38): and 2) this suppression is reversed by membrane recruitment of Rap1GAP, a GTPase which also acts as a GDI that binds via its GoLoco motif to the α-subunit of Gi protein, thereby inhibiting Rap1 activity [36-41]. Thus, whereas Rap1 activation may be viewed as a homeostatic “breaking” mechanism that counteracts Gβγ-coupled/c-Src-stimulated Ras activity, membrane recruitment of Rap1GAP serves to suppress the latter homeostatic action of Rap1 [39-41]. Accordingly, Gi protein activation leading to Gβγ-induced c-Src stimulation and Gα-coupled recruitment of Rap1GAP to inactivate Rap1 can be seen as acting cooperatively to heighten Ras stimulation of the MEK-ERK1/2 cascade, as previously proposed [39]. Our data herein support this notion and, further, that these Gi-regulated cooperative signaling events are constitutively activated in asthmatic HASM cells, given that the intrinsically heightened Gβγ-induced ERK1/2-stimulated PDE activity is abrogated by inhibition of c-Src signaling (FIG. 4), and is significantly attenuated by inhibition of Rap1GAP using targeted siRNA preparations (FIG. 6). In this context, it is of interest to note that activation of PKA in striatal neurons was recently shown to phosphorylate Rap1GAP and, thereby inhibit its GAP activity, resulting in an increase in Rap1 activity [49]. A priori, if also present in HASM cells, such an effect of PKA activation would be expected to attenuate Gβγ-regulated c-Src/Ras-mediated ERK1/2 activation. This possibility raises the consideration that PKA activation (e.g., using cAMP-elevating agents) may act synergistically with an inhibitor of Gβγ-induced c-Src activation to suppress the pro-asthmatic state in asthmatic HASM cells. This feasible consideration warrants systematic investigation.

In further considering the present observations, it should be noted that, unlike the sustained suppressive effect of treatment with anti-Gβγ blocking peptide on PDE activity in asthmatic HASM cells, the inhibitory effect of gallein was transient in nature (FIG. 1). Of interest, a disparity between these Gβγ inhibitors was also observed in our previous study wherein, unlike pretreatment with the anti-Gβγ blocking peptide which inhibited both the in vivo AHR and pulmonary inflammation elicited by allergen challenge in allergic rabbits, pretreatment with gallein only suppressed AHR and had no appreciable inhibitory effect on pulmonary inflammation (18). We speculated that, while the disparity between these inhibitors is not readily explained, possible explanations include differences in their pharmacodynamic or pharmacokinetic properties that might influence their inhibitory actions on Gβγ-regulated effector systems, and/or differences in their respective mechanisms of Gβγ inhibition [18]. Regarding the latter possibility, as previously reported [50], given that gallein is a small molecule with a distinctive spacial orientation of binding to the bioactive “hot spot” on the Gβγ surface, the nature and extent of its inhibition of Gβγ-targeted effector interactions may be relatively limited. Conversely, by sequestering the Gβγ subunit, the anti-Gβγ blocking peptide is arguably capable of relatively greater and more effective inhibition of Gβγ interactions with various effector targets [18]. These interesting considerations are worthy of systematic investigation.

In evaluating the implications of the present findings, it must be emphasized that the data pertain to studies conducted using a relatively small number of commercially available cultured HASM cell lines, with little history available regarding the asthmatic HASM cell donors (Table 1). Thus, the extent to which our observations pertain to the in vivo human condition is open to speculation. In this regard, however, it is noteworthy that, notwithstanding the limited number of asthmatic cell lines examined, our results concur with those in previous studies, including the aforementioned study that also reported significantly increased intrinsic PDE4 activity in cultured HASM cells isolated from asthmatic individuals relative to normal non-asthmatic HASM cells [27], as well as the studies that implicated upregulated PDE4 activity in mediating the in vivo airway responses to allergen challenge in asthmatic individuals [19,21] and in animal models of allergic asthma [18,22-26]. This concurrence of findings is further substantiated when considering that the present observations are compatible with those in previous reports wherein Gβγ signaling coupled to ERK1/2 activation was shown to critically regulate the increased PDE4 activity experimentally induced in cultured normal HASM cells and isolated rabbit ASM tissues exposed to pro-asthmatic stimulation with either IL-13, atopic asthmatic serum, or long-acting β2AR agonists, as well as the corresponding PDE4-mediated pro-asthmatic changes in contractility exhibited by the stimulated ASM tissues [16-18,32]. In view of these considerations, we believe that the findings of the present study are likely applicable to the human asthmatic vs. normal in vivo condition.

In conclusion, this study is the first to report that intrinsically increased signaling by the Gβγ subunit, facilitated by recruitment of Rap1GAP to the Gαi-subunit, enables heightened Src-induced ERK1/2 activation and its consequent direct induction of the constitutively increased PDE4 activity exhibited in asthmatic HASM cells. To the extent that upregulated PDE4 activity is known to play a pivotal role in mediating the airway asthmatic phenotype, the present findings indicated that suppressing Gβγ function provides a novel approach to treat asthma. In this context, the data provided appear to indicate that suppression of Gβγ function leading to increased PDE4 activity in asthmatic HASM cells was most effectively achieved by treatment with the anti-Gβγ blocking peptide, resulting in essentially complete PDE4 suppression (FIG. 1). By comparison, the Gβγ-regulated heightened PDE4 activity was suppressed to a notably lesser, albeit significant, extent (i.e., ˜40%) by treatment with the Rap1GAP siRNA preparation (FIG. 6B). This observation is not surprising given that Rap1GAP plays a modulatory rather than decisive role in regulating signaling downstream to Gβγ activation (39). Notwithstanding this consideration, given our present findings, Rap1GAP inhibition may be therapeutically beneficial in asthma, whether used alone or in combination with anti-Gβγ or other conventional therapies. In this regard, it is noteworthy that cAMP-induced PKA activation was previously reported to phosphorylate and, thereby, inhibit Rap1GAP activity in central striatal neurons (51). To the extent that cAMP-elevating agents (notably, β-adrenergic agonists) are commonly used to treat asthma, it is conceivable that their therapeutic efficacy in asthma management may be explained, at least in part, by inhibition of Rap1GAP activity in the airways. This intriguing possibility is worthy of future investigation. Notably, a significant number of asthmatic individuals can be adversely affected by treatment with β-adrenergic or other cAMP-elevating agents (e.g., beta agonists). Such patients may benefit from the nucleic acid based approaches described herein for inhibition of Rap1GAP activity. In preferred approaches the agent inhibits Rap1GAP activity directly and does not act on an upstream modulator.

### REFERENCES

1. Druey K M (2009) Regulation of G-protein-coupled signaling pathways in allergic inflammation. Immunol Res 43:62-76.
2. Hepler J R, Gilman A G (1992) G proteins. Trends Biochem Sci 17: 383-387.
3. Goldsmith Z B, Dhanasekaran D N (2007) G protein regulation of MAPK networks. Oncogene 26: 3122-3142.
4. Gerthoffer W T, Singer C A (2003) MAPK regulation of gene expression in airway smooth muscle. Respir Physiol Neurobiol 137: 237-250.
5. Hakonarson H, Grunstein M M (2003) Autocrine regulation of airway smooth muscle responsiveness. Respir Physiol Neurobiol 137: 263-276.
6. Billington C K, Penn R B (2003). Signaling and regulation of G protein-coupled receptors in airway smooth muscle. Respir Res. 4:2 Epub 2003: 1-28.
7. Hershenson M B, Brown M, Camoretti-Mercado B, Solway J (2008) Airway smooth muscle in asthma. Annu Rev Pathol Mech Dis 3: 523-555.
8. Stork P J, Schmitt J M (2002) Crosstalk between cAMP and MAP kinase signaling in the regulation of cell proliferation. Trends Cell Biol 12:258-266.
9. Crespo P, Xu N, Simonds W F, Gutkind J S (1994) Ras-dependent activation of MAP kinase pathway mediated by G-protein G βγ subunits. Nature 369:418-420.
10. Koch W J, Hawes B E, Allen L F, Lefkowitz R J (1994) Direct evidence that Gi-coupled receptor stimulation of mitogen-activated protein kinase is mediated by G βγ activation of p21ras. Proc Natl Acad Sci USA 91:12706-12710.
11. Luttrell L M, Hawes B E, van Biesen T, Luttrell D K, Lansing T J, et al. (1996) Role of c-Src tyrosine kinase in G protein-coupled receptor- and G βγ subunit-mediated activation of mitogen-activated protein kinases. J Biol Chem 271: 19443-19450.
12. McCudden C R, Hains M D, Kimple R J, Siderovski D P, Willard F S (2005) G-protein signaling: back to the future. Cell Mol Life Sci 62: 551-577.
13. Hakonarson H, Herrick D J, Grunstein M M (1995) Mechanism of impaired β-adrenoceptor responsiveness in atopic sensitized airway smooth muscle. Am J Physiol Lung Cell Mol Physiol 269: L645-L652.
14. Hakonarson H, Herrick D J, Gonzalez Serrano P, and Grunstein M M (1996) Mechanism of cytokine-induced modulation of β-adrenoceptor responsiveness in airway smooth muscle. J Clin Invest 97: 2593-2600.
15. Hakonarson H, Maskeri N, Carter C, Hodinka R L, Campbell D, et al. (1998) Mechanism of rhinovirus-induced changes in airway smooth muscle responsiveness. J Clin Invest 102: 1732-1741.
16. Hu A, Nino G, Grunstein J S, Fatma S, Grunstein M M (2008) Prolonged heterologous □2-adrenoceptor desensitization promotes proasthmatic airway smooth muscle function via PKA/ERK1/2-mediated phosphodiesterase-4 induction. Am J Physiol Lung Cell Mol Physiol 294:L1055-L1067.
17. Nino G, Hu A, Grunstein J S, Grunstein M M (2009) Mechanism regulating proasthmatic effects of prolonged homologous β2-adrenergic receptor desensitization in airway smooth muscle. Am J Physiol Lung Cell Mol Physiol 297: L746-L757.
18. Nino G, Hu A, Grunstein J S, McDonough J, Kreiger P A, Josephson M B, Choi J K,
Grunstein M M (2012). G protein βγ-subunit signaling mediates airway hyperresponsiveness and inflammation in allergic asthma. PLoS One. 7(2): Epub 2012.
19. van Schalkwyk E, Strydom K, Williams Z, Venter L, Leichtl S, et al. (2005) Roflumilast, an oral, once-daily phosphodiesterase 4 inhibitor, attenuates allergen-induced asthmatic reactions. J Allergy Clin Immunol 116: 292-298.
20. Bateman E D, Izquierdo J L, Harvest U, Hofbauer P, Magyar P, et al. (2006) Efficacy and safety of roflumilast in the treatment of asthma. Ann Allergy Asthma Immunol 96: 679-686.
21. Louw C, Williams Z, Venter L, Leichtl S, Schmid-Wirlitsch C, et al. (2007) Roflumilast, a phosphodiesterase 4 inhibitor, reduces airway hyperresponsive-ness after allergen challenge. Respiration 74: 411-417.
22. Hansen G, Jin S, Umetsu D T, Conti M (2000) Absence of muscarinic cholinergic airway responses in mice deficient in the cyclic nucleotide phosphodiesterase PDE4D. Proc Natl Acad Sci USA 97: 6751-6756.
23. Kanehiro A, Ikemura T, Makela M J, Lahn M, Joetham A, et al. (2001) Inhibition of phosphodiesterase 4 attenuates airway hyperresponsiveness and airway inflammation in a model of secondary allergen challenge. Am J Respir Crit Care Med 163: 173-184.
24. Tang H F, Song Y H, Chen J C, Chen J Q, Wang P (2005) Upregulation of phosphodiesterase-4 in the lung of allergic rats. Am J Respir Crit Care Med 171:823-828.
25. Sun J G, Deng Y M, Wu X, Tang H F, Deng J F, et al. (2006) Inhibition of phosphodiesterase activity, airway inflammation and hyperresponsiveness by PDE4 inhibitor and glucocorticoid in a murine model of allergic asthma. Life Sci 79: 2077-2085.
26. Chapman R W, House A, Jones H, Richard J, Celly C, et al. (2007) Effect of inhaled roflumilast on the prevention and resolution of allergen-induced late phase airflow obstruction in Brown Norway rats. Eur J Pharmacol 571: 215-221.
27. Trian T, Burgess J K, Niimi K, Moir L M, Ge Q, Berger P, Liggett S B, Black J L, Oliver B G (2011). β2-Agonist induced cAMP is decreased in asthmatic airway smooth muscle due to increased PDE4D. PLoS One. 6(5):e20000.
28. Chang M, Zhang L, Tam J P, Sanders-Bush E (2000) Dissecting G protein-coupled receptor signaling pathways with membrane-permeable blocking peptides. J Biol Chem 275: 7021-7029.
29. Casey L M, Pistner A R, Belmonte S L, Migdalovich D, Stolpnik O, et al. (2010) Small molecule disruption of G βγ signaling inhibits the progression of heart failure. Circ Res 107: 532-539.
30. Lehmann D M, Seneviratne A M, Smrcka A V (2008) Small molecule disruption of G protein βγ subunit signaling inhibits neutrophil chemotaxis and inflammation. Mol Pharmacol 73:410-418.
31. Hu A, Fatma S, Cao J, Grunstein J S, Nino G, Grumbach Y, and Grunstein M M (2009) Th2 cytokine-induced upregulation of 11beta-hydroxysteroid dehydrogenase-1 facilitates glucocorticoid suppression of proasthmatic airway smooth muscle function. Am J Physiol Lung Cell Mol Physiol 296: L790-803.
32. Nino G, Hu A, Grunstein J S, Grunstein M M (2010) Mechanism of glucocorticoid protection of airway smooth muscle from proasthmatic effects of long-acting beta2-adrenoceptor agonist exposure. J Allergy Clin Immunol 125:1020-1027.
33. Kmiecik T E, Shalloway D (1987) Activation and suppression of pp60c-src transforming ability by mutation of Its primary sites of tyrosine phosphorylation. Cell 49: 65-73.\
34. Billington C K, Le Jeune I R, Young K W, Hall I P (2008) A major functional role for phosphodiesterase 4D5 in human airway smooth muscle cells. Am J Respir Cell Mol Biol 38: 1-7.
35. Liu H, Maurice D H (1999) Phosphorylation-mediated activation and translocation of the cyclic AMP-specific phosphodiesterase PDE4D3 by cyclic AMP-dependent protein kinase and mitogen-activated protein kinases. A potential mechanism allowing for the coordinated regulation of PDE4D activity and targeting. J Biol Chem 274:10557-10565.
36. Kitayama, H. Sugimoto Y, Matsuzaki T, Ikawa Y, Noda M (1989) A ras-related gene with transformation suppressor activity. Cell 56: 77-84.
37. Cook S J, Rubinfeld B, Albert I, McCormick F (1993) RapV12 antagonizes Ras-dependent activation of ERK1 and ERK2 by LPA and EGF in Rat-1 fibroblasts. EMBO J. 12: 3475-3485.
38. Okada, S. Matsuda M, Anafi M, Pawson T, Pessin J E (1998) Insulin regulates the dynamic balance between Ras and Rap1 signaling by coordinating the assembly states of the Grb2-SOS and CrkII-C3G complexes. EMBO J 17: 2554-2565.
39. Mochizuki N, Ohba Y, Kiyokawa E, Kurata T, Murakami T, Ozaki T, Kitabatake A, Nagashima K, Matsuda M (1999) Activation of the ERK/MAPK pathway by an isoform of rap1GAP associated with G alpha(i). Nature. 400: 891-894.
40. Willard F S, Low A B, McCudden C R, Siderovski D P (2007) Differential G-alpha interaction capacities of the GoLoco motifs in Rap GTPase activating proteins. Cell Signal 19: 428-438.
41. van Dam T J, Bos J L, Snel B (2011) Evolution of the Ras-like small GTPases and their regulators. Small GTPases. 2: 4-16.
42. Shu F J, Ramineni S, Hepler J R (2010) RGS14 is a multifunctional scaffold that integrates G protein and Ras/Raf MAPkinase signalling pathways. Cell Signal 22: 366-376.
43. Houslay M D, Adams D R. (2003) PDE4 cAMP phosphodiesterases: modular enzymes that orchestrate signalling cross-talk, desensitization and compartmentalization. Biochem J 370: 1-18.
44. Stork P J, Schmitt J M (2002) Crosstalk between cAMP and MAP kinase signaling in the regulation of cell proliferation. Trends Cell Biol 12:258-266.
45. Crespo P, Xu N, Simonds W F, Gutkind J S (1994) Ras-dependent activation of MAP kinase pathway mediated by G-protein G βγ subunits. Nature 369:418-420.
46. Koch W J, Hawes B E, Allen L F, Lefkowitz R J (1994) Direct evidence that Gi-coupled receptor stimulation of mitogen-activated protein kinase is mediated by G βγ activation of p21ras. Proc Natl Acad Sci USA 91:12706-12710.
47. Luttrell L M, Hawes B E, van Biesen T, Luttrell D K, Lansing T J, et al. (1996) Role of c-Src tyrosine kinase in G protein-coupled receptor- and Gβγ subunit-mediated activation of mitogen-activated protein kinases. J Biol Chem 271: 19443-19450.
48. McCudden C R, Hains M D, Kimple R J, Siderovski D P, Willard F S (2005) G-protein signaling: back to the future. Cell Mol Life Sci 62: 551-577.
49. Smrcka A V (2008) G protein βγ subunits: Central mediators of G protein-coupled receptor signaling. Cell Mol Life Sci 65:2191-2214.
50. McAvoy T, Zhou M M, Greengard P, Nairn A C (2009) Phosphorylation of Rap1GAP, a striatally enriched protein, by protein kinase A controls Rap1 activity and dendritic spine morphology. Proc Natl Acad Sci USA. 106:3531-3536.
51. McAvoy T, Zhou M M, Greengard P, Nairn A C. (2009) Phosphorylation of Rap1GAP, a striatally enriched protein, by protein kinase A controls Rap1 activity and dendritic spine morphology. Proc Natl Acad Sci USA. 106:3531-3536.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

1. A method for the treatment of asthma in a patient in need thereof, comprising administration of an effective amount of an inhibitor of Rap1GAP activity, said administration being effective to reduce bronchoconstriction in said patient.

2. The method of claim 1 wherein said composition is delivered to a patient by a method selected from the group comprising, systemic, oral, intravenous, intramuscular, subcutaneous, intraorbital, intranasal, intracapsular, intraperitoneal, intracisternal, intratracheal, intraarticular administration, or by absorption through the skin to inhibit constriction of airway smooth muscle in the lung.

3. The method of claim 1, wherein said inhibitor of Rap1GAP is formulated for aerosol delivery and is delivered via inhalation.

4. The method of claim 3, further comprising administration of at least one anti-inflammatory agent selected from the group consisting of corticosteroids, sodium cromolyn, IgE inhibitors, phosphodiesterase inhibitors, methylxanthines, beta-adrenergic agents, and leukotriene modifiers.

5. The method of claim 4, wherein said agent is delivered simultaneously with said inhibitor.

6. The method of claim 4, wherein said agent is delivered sequentially, before or after delivery of said inhibitor.

7. The method of claim 1, wherein said inhibitor of Rap1Gap activity is a siRNA directed to Rap1Gap encoding RNA.

8. The composition of claim 3, wherein said aerosolized formulation comprises a propellant selected from the group consisting of halocarbons, hydrocarbons and esters.

9. The method of claim 1, wherein said inhibitor is a nucleic acid directed to Rap1GAP encoding mRNA that interferes with expression of Rap1GAP protein.

10. The method of claim 9, wherein said inhibitor is an siRNA.

11. The method of claim 1, where said inhibitor is not a beta adrenergic elevating agent.

12. The method of claim 1, where said inhibitor is not a cAMP elevating agent.

13. The method of claim 3, wherein said inhibitor is a nucleic acid directed to Rap1GAP encoding mRNA that interferes with expression of Rap1GAP protein.

14. The method of claim 3, where said inhibitor is not a beta adrenergic elevating agent.

15. The method of claim 3, where said inhibitor is not a cAMP elevating agent.
